(19) United States
(12) Patent Application Publication
(10) Pub. No.: US 2013/0028838 A1
(43) Pub. Date: Jan. 31, 2013

(54) F18-TYROSINE DERIVATIVES FOR IMAGING BONE METASTASES

(71) Applicants: Sabine Zitzmann-Kolbe, Berlin (DE); Keith Graham, Berlin (DE)

(72) Inventors: Sabine Zitzmann-Kolbe, Berlin (DE); Keith Graham, Berlin (DE)

(73) Assignee: PIRAMAL IMAGING SA, Matran (CH)

(21) Appl. No.: 13/577,454

(22) PCT Filed: Feb. 4, 2011
(86) PCT No.: PCT/EP11/51636

§ 371 Date: Oct. 15, 2012

(30) Foreign Application Priority Data
Feb. 8, 2010 (EP) 10075055.3

(51) Int. Cl. A61K 51/04; C07C 229/36

(52) U.S. Cl. 424/1.89; 562/445

(57) ABSTRACT

This invention relates to radioactive tyrosine derivatives for imaging bone metastases, a method for imaging or diagnosing bone metastases, compositions and kits for imaging bone metastases.

### FIELD OF INVENTION

The invention relates to radioactive tyrosine derivatives for imaging bone metastases, a method for imaging or diagnosing bone metastases, compositions and kits for imaging bone metastases.

### BACKGROUND

Amino acids are important biological substrates, which play crucial roles in virtually all biological processes. They accumulate in malignant transformed cells due to increased expression of amino acid transporters, which are essential for the growth and proliferation of normal and transformed cells (Christensen H N. Role of amino acid transport and counter transport in nutrition and metabolism. Physiol Rev. January 1990; 70(1):43-77). One important amino acid transporter is the L-type amino acid transporter 1 (LAT1), which transports large neutral amino acids such as leucine, isoleucine, valine, phenylalanine, tyrosine, tryptophan, methionine, and histidine (Yanagida O, Kanai Y, Chairoungdua A, et al. Human L-type amino acid transporter 1 (LAT1): characterization of function and expression in tumor cell lines. Biochim Biophys Acta. Oct. 1 2001; 1514(2):291-302). Localization studies and functional in vitro and in vivo data suggest that LAT1 is physiologically essential for the (directional) import of amino acids into growing cells. There is evidence that LAT1 uses intracellular amino acid concentrations generated by other transporters, in particular amino acid transporter ASCT2 (SLC1A5) seems to play a role, to exchange these amino acids for other essential amino acids (Fuchs B C, Bode B P. Amino acid transporters ASCT2 and LAT1 in cancer: partners in crime? Semin Cancer Biol. August 2005; 15(4):254-266).

LAT1 protein is highly expressed in many tumors and tumor cell lines of various origins (Kobayashi H, Ishii Y, Takayama T. Expression of L-type amino acid transporter 1 (LAT1) in esophageal carcinoma. J Surg Oncol. Jun. 15 2005; 90(4):233-238 and Nawashiro H, Otani N, Shinomiya N, et al. L-type amino acid transporter 1 as a potential molecular target in human astrocytic tumors. Int J. Cancer. Aug. 1 2006; 119(3):484-49). In a study with 321 patients investigating lung tumors, 29% of adenocarcinoma, 91% squamous cell carcinoma and 67% large cell carcinoma were positive for LAT1 protein expression and the expression correlated positively with the proliferation marker Ki-67 (Kaira K, Oriuchi N, Imai H, et al. Prognostic significance of L-type amino acid transporter 1 expression in resectable stage I-III non small cell lung cancer. Br J. Cancer. Feb. 26 2008; 98(4):742-748). Various tyrosine derivatives have been labeled with F-18 to make use of the L-transporter system for positron emission tomography (PET) tumor imaging.

Urakami et al. (Nuclear Medicine and Biology 36 (2009) 295-303) have demonstrated that F18 labeled D and L-Fluoro methyl tyrosines (D-[18F]FMT/L-[18F]FMT) are accumulated into tumor cells via amino transporter. The inoculated tumor cells in tumor-bearing mice are HeLa cells and C6 glioma cells. The mouse injected with D-[18F]FMT showed the clearest difference in tracer intensity between the tumor (right leg) and the normal tissue (left leg) compared with the mice given F18-fluorodeoxyglucose (F18-FDG) tracer. D-[18F]FMT was found to be a potential tumor-detecting agent for PET, especially for the imaging of a brain cancer and an abdominal cancer.

Bone is a the site of cancer wherein the cancer can be in the form of a malignant tumor characterized by abnormal growth of cells or of cancerous metastasis resulting from tumor spreading to other locations in the body such as bone via lymph or blood. Metastatic bone disease from solid tumors often poses significant problems for the oncologist, usually mandating a radical change to the therapeutic approach, and is particularly important for minimizing the risk of pathologic fracture (Chua S et al, Semin Nucl Med 2009, 39:416-430). Bone Scintigraphy using technetium-labeled diphosphonates has long been the mainstay of functional imaging of bone metastases, but has the limitation of relatively poor specificity. It relies on detection of abnormal osteoblastic response elicited by the malignant cells. Bone scintigraphy offers the advantage of total body examination, low cost, and mostly a high degree of sensitivity. The major limitation of scintigraphy is its lack of specificity; many benign bone pathologies produce a hot spot on scintigraphy, which may not be distinguishable from a metastasis. SPECT has been shown to significantly improve the predictive value of bone scintigraphy, and although SPECT accuracy is significantly higher than that of planar scintigraphy, there is still room for improvement of anatomic localization and characterization.

PET can achieve a higher spatial resolution than that of single photon imaging, a factor that can be particularly helpful in interpreting subtle bone lesions. F18-FDG has been reported to be appropriate for detecting all types of bone metastases. However, the accuracy of FDG PET imaging was questioned by Even-Sapir et al. (Seminars in musculoskeletal radiology vol 11, 4 2007). Indeed, it was found that for some patients the FDG PET imaging is not concordant with Computed Tomography (CT). Taira et al. (Radiology vol 243 1 Apr. 2007, 204) stipulates that FDG-PET/CT has a very high positive predictive value (PPV) for bone metastases (98%) when the findings at PET and CT are concordant; however, in lesions with discordant PET and CT findings at the integrated examination, PPV is markedly diminished. A drawback is that the uptake of the main tracer used, namely, 18F-fluorodeoxyglucose (18F-FDG), is dependent on the higher glycolytic rates of most tumors compared with normal tissues. This reduces the sensitivity of PET in the detection of metastases of slowgrowing tumors, such as carcinoid tumors. It does, however, mean that uptake is directly dependent on the presence of tumor cells rather than the osteoblastic bone reaction as in the case of bone scanning, so that unlike the latter it can play a valuable role in myeloma.

[F-18]-fluoride is known also as a PET bone-seeking agent, because [F-18]-fluoride is incorporating into Apatite molecules in exchange for a hydroxy-group (Schirrmeister H et al. Detection of bone metastases in breast cancer by positron emission tomography. Radio) Clin North Am. 45(4):669-676). Thus, [F-18]-fluoride reflects an unspecific uptake into regenerating and remineralizing bone. Park-Holohan et al. (Nuclear Medicine Communications, 2001 September (22)₉, 1037) evaluate the skeletal kinetic of two tracers [F-18]-fluoride and 99 mTc-methylene diphosphonate reflecting bone blood flow and osteoblastic activity. It was observed that approximately 30% of [F-18]-fluoride blood-borne tracer is carried in red cells suggesting that red cell [F-18]-fluoride is largely available for uptake in bone. In contrast to [F-18]-fluoride, the red cell concentrations of 99 mTc-MDP was found to be negligibly small. [F-18]-fluoride is distributed and taken up in the whole body bones as well in bone metastases with a high metastase-bone ratio. The major limitation, however, is the same as for technetium-labeled diphosphonates. There is a lack of specificity; which does not allow the differentiation of many benign bone pathologies from a metastasis.

There a clear need for an accurate PET tracer for imaging bone metastases wherein uptake is specific in bone metastatses.

It was surprisingly found that [F-18]-tyrosine derivatives PET tracers such as [F-18]-D-FMT that are useful for imaging bone metastases.

### SUMMARY

In a first aspect, the invention is directed to a radioactive tyrosine derivatives of general formula (I) for imaging bone metastases. In a second aspect, the invention is directed to the use of compound of formula (I) for differentiating bone metastatic disease from bone non-metastatic disease in mammal. In a third and fourth aspects, the invention is directed to a composition or a kit comprising radioactive tyrosine derivatives of the general formula (I), (D-I), or mixture thereof and pharmaceutically acceptable carrier or diluent wherein the compounds of the general formula (I), (D-I) are imaging tracer for imaging bone metastases.

### DRAWINGS

FIG. 1: PET/CT images of [F-18]-D-FMT and [F-18]-fluoride from a mouse with 786-O bone metastases. The scans were performed 2 weeks apart, first the [F-18]-fluoride scan and then the D-FMT scan. D-FMT accumulates into tumor cells, [F-18]-fluoride is incorporated into regenerating bone. Grey arrows indicate some of the metastases.

FIG. 2: PET/CT images of [F-18]-D-FMT from a mouse with 786-O bone metastases (left image CT, middle image PET, right image PET/CT fusion image). CT images were calculated using surface rendering program. Images shows dorsal view. Grey arrows indicate some of the metastases.

FIG. 3: PET/CT images of [F-18]-D-FMT and [F-18]-fluoride from a mouse with 786-O bone metastases and the corresponding histopathological lesions (H&E). Hematopoietic cell areas are wholly replaced by tumor tissue in the medullary cavity. B shows an area with large tumor cells and in C the tumor is composed of spindle cells. In D there is an area of hematopoietic cells still present (*) beside the tumor mass (T). In E lysis of normal bone occurred simultaneously with the formation of osteoid (E-1, H&E), which stained blue green with MTG (E-2). The tumor cells were positive for pan-cytokeratin (E-3). In F the tumor cells replace the haematopoietic cells with lysis of normal bone (F-1, H&E; F-2). The tumor cells were positive for pan cytokeratin (F-3).

FIG. 4: PET/CT images of [F-18]-D-FMT from mice with MDA-MB231SA bone metastases. The scans were performed 25 days after the inoculation. D-FMT accumulates into tumor cells delineating sites of bone metastases formation. Grey arrows indicate some of the metastases.

### DESCRIPTION

In a first aspect, the invention is directed to compounds of general formula (I) for imaging bone metastases wherein

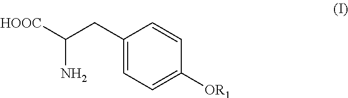

R₁ is —CH₂—F¹⁸, —CH₂—CH₂—F¹⁸ or —CH₂—CH₂—CH₂—F¹⁸ and pharmaceutically acceptable salts thereof.

Invention encompasses also the single isomers, enantiomers, stereoisomers, stereoisomeric mixtures or mixtures of compounds of general formula (I).

Preferably, the invention is directed to compounds of general formula (I) for imaging bone metastases wherein

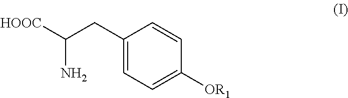

R₁ is —CH₂—F¹⁸ or —CH₂—CH₂—F¹⁸ and pharmaceutically acceptable salts thereof.

In other word, the invention is directed to the use of compounds of general formula (I) for the manufacture of an imaging tracer for imaging bone metastases wherein

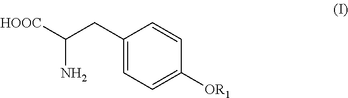

R₁ is —CH₂—F¹⁸, —CH₂—CH₂—F¹⁸, or —CH₂—CH₂—CH₂—F¹⁸ and pharmaceutically acceptable salts thereof.

The invention is directed to compound of general formula (I) for use in the imaging bone metastases.

Preferably, the compound of formula (I) is a D-tyrosine derivative of formula (D-I)

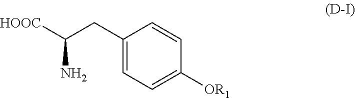

wherein R₁ is —CH₂—F¹⁸, —CH₂—CH₂—F¹⁸, or —CH₂—CH₂—CH₂—F¹⁸.

More preferably, the compound of formula (I) is a D-tyrosine derivative of formula (D-I)

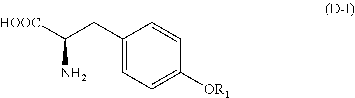

wherein R₁ is —CH₂—F¹⁸, or —CH₂—CH₂—F¹⁸.

Even more preferably, the compound is

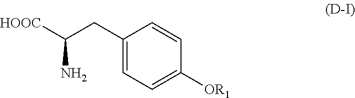

wherein R₁ is —CH₂—F¹⁸ and
named (R)-2-amino-3-(4[F-18]fluoromethoxy-phenyl)-propionic acid=

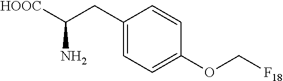

The invention is directed to compound of general formula (D-I) or R)-2-amino-3-(4-[F-18]fluoromethoxy-phenyl)-propionic acid for use in the imaging bone metastases.

The imaging tracer is suitable for Positron Emission Tomography (PET) or MicroPET.

The imaging comprises the step of PET imaging and is optionally preceded or followed by a Computed Tomography (CT) imaging or Magnetic Resonance Tomography (MRT) imaging. The imaging occurs in mammals.

The invention is also directed to a method for imaging or diagnosis bone metastases comprising the steps:
Administering to a mammal an effective amount of compounds of general formula (I) or (D-I) or mixture there of,
Obtaining images of the mammal and
Assessing the images.

Preferably, the invention concerns, compound of formula

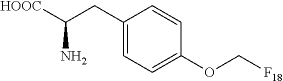

and pharmaceutically acceptable salts thereof for the manufacture of an imaging tracer for imaging bone metastases.

In a second aspect, the invention is directed to the use of compound of formula (I) for differentiating bone metastatic disease from bone non-metastatic disease in mammal. Preferred embodiments disclosed above in respect of compound of formula (I) are included herein.

The invention is also directed to a method for differentiating bone metastatic disease from bone non-metastatic disease in mammal by assessing image(s) obtained after administering to the mammal of an effective amount of compounds of general formula (I) or (D-I) or mixture there of.

Bone non-metastatic diseases are benign bone pathologies comprised from the group of back pains, focal changes in bones, trauma, reconstructive surgery, bone grafts, metabolic bone disease or osteoporosis.

In a third aspect, the invention is directed to a composition comprising compounds of the general formula (I), (D-I), or mixture thereof and pharmaceutically acceptable carrier or diluent wherein the compounds of the general formula (I), (D-I) are imaging tracer for imaging bone metastases.

The person skilled in the art is familiar with auxiliaries, vehicles, excipients, diluents, solvents, carriers or adjuvants which are suitable for the desired pharmaceutical formulations, preparations or compositions on account of his/her expert knowledge. The administration of the compounds, pharmaceutical compositions or combinations according to the invention is performed in any of the generally accepted modes of administration available in the art. Intravenous deliveries are preferred.

Generally, the compositions according to the invention is administered such that the dose of the active compound for imaging is in the range of 37 MBq (1 mCi) to 740 MBq (20 mCi). In particular, a dose in the range from 150 MBq to 370 MBq will be used.

In a fourth aspect, the present invention provides a kit comprising a sealed vial containing a predetermined quantity of a compound having general chemical Formula (I) or (D-I) and suitable salts of inorganic or organic acids thereof, hydrates, complexes, esters, amides, and solvates thereof for imaging bone metastases.

Optionally the kit comprises a pharmaceutically acceptable carrier, diluent, excipient or adjuvant.

### DEFINITIONS

The terms used in the present invention are defined below but are not limiting the invention scope.

Suitable salts of the compounds according to the invention include salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalene disulphonic acid, acetic acid, trifluoroacetic acid, propionic acid, lactic acid, tartaric acid, malic acid, citric acid, fumaric acid, maleic acid and benzoic acid.

Suitable salts of the compounds according to the invention also include salts of customary bases, such as, by way of example and by way of preference, alkali metal salts (for example sodium salts and potassium salts), alkaline earth metal salts (for example calcium salts and magnesium salts) and ammonium salts, derived from ammonia or organic amines having 1 to 16 carbon atoms, such as, by way of example and by way of preference, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

Unless otherwise specified, when referring to the compounds of formula the present invention per se as well as to any pharmaceutical composition thereof the present invention includes all of the hydrates, salts, and complexes.

As used herein, the term “carrier” refers to microcrystalline cellulose, lactose, mannitol.

As used herein, the term “solvents” refers to liquid polyethylene glycols, ethanol, corn oil, cottonseed oil, glycerol, isopropanol, mineral oil, oleic acid, peanut oil, purified water, water for injection, sterile water for injection and sterile water for irrigation.

### EXPERIMENTAL PART

### Abbreviations

| | |
|---|---|
| DMF | N,N-dimethylformamide |
| DMSO | Dimethylsulfoxide |
| HPLC | high performance liquid chromatography |
| GBq | Giga Bequerel |
| MBq | Mega Bequerel |

In this study, it was investigated the potential of D-FMT to image bone metastases in two mouse models. Injection of 786-O/luc cells and MDA-MB231SA/luc cells into the arterial circulation resulted in the development of aggressive osteolytic lesions in bones within 62±8 days for the 786-O/luc cells and 20±5 days for the MDA-MB231SA/luc cells. Due to the variety of cytokines and growth factors stored in bone, the skeleton provides a fertile environment for the growth of cancer cells (13). The tumor cells were primarily located within the bone and resulted in cortical destruction of bone. No soft tissue metastases (kidneys, adrenal glands, heart, lungs) were detected by bioluminescence imaging or by histomorphometry (14). A bone scan with [F-18]-fluoride was performed to validate the localization of the bone metastases.

### Material and Methods

### Cell Lines

The 786-O/luciferase (luc) cell line was generated by stable transfection with a pRev CMV_luc2 vector. The cells were cultured in RPMI medium (Biochrom AG, Berlin, Germany) containing 10% heat-inactivated FCS (Biochrom AG), 2% glutamine (PPA Laboratories, Pasching, Austria), 4.5 g/l glucose (Sigma-Aldrich Chemie GmbH, Taufkirchen, Germany), 10 mM HEPES (Biochrom AG), 1 mM Pyruvate (Biochrom AG) and 50 μg/ml hygromycin B (Invitrogen Ltd; Carlsbad, Calif., USA).

The MDA-MB231/luciferase (luc) cell line was generated by stable transfection with a pRev CMV_luc2 vector. Cells were cultivated in high-glucose DMEM (Biochrom AG) containing 10% heat-inactivated FCS (Biochrom AG), 2% glutamine (PAA Laboratories GmbH), 1% nonessential amino acids (PAA Laboratories) and 250 μg/mL hygromycin B (Invitrogen Ltd.).

### Animals and Tumor Cell Growth

786-O/luc cells and the MDA-MB231SA/luc cells were harvested from subconfluent cell culture flasks and resuspended in PBS (Biochrom AG) to a final concentration of 5×10⁵ cells/100 μl. For intracardiac inoculations, 5-week-old female athymic nude mice (Harlan-Winkelmann GmbH, Borchen, Germany) were anesthetized with an intraperitoneal injection of 5% Rompun (Bayer HealthCare AG, Leverkusen, Germany)/10% Ketavet (Pfizer, Karlsruhe, Germany) in 0.9% NaCl at a dose of 0.1 ml/10 g body weight. Using an insulin syringe (BD Micro-Fine+Demi U-100, Becton Dickinson GmbH, Heidelberg, Germany), 5×10⁵ 786-O/luc cells in 100 μl PBS were inoculated into the left cardiac ventricle (i.c.) of anesthetized mice. Experiments were approved by the governmental review committee on animal care.

### Optical Imaging

Tumor cell dissemination in bone was regularly monitored by bioluminescence imaging using a cooled CCD camera (NightOWL LB, Berthold Technologies, Bad Wildbad, Germany). The mice were injected intravenously with 100 μl luciferin (45 mg/ml in PBS, Synchem OHG, Felsberg/Altenburg, Germany) and anesthetized with 1-3% isoflurane (CuraMED Pharma GmbH, Karlsruhe, Germany).

Radiosynthesis of D-[F-18]-Fluoromethyl tyrosine (D-FMT)

The synthesis of D-[F-18]-fluoromethyl tyrosine (D-FMT) was performed by reacting [F-18]-fluoromethyl bromide with D-Tyrosine as previously described by Tsukada H, Sato K, Fukumoto D, Nishiyama S, Harada N, Kakiuchi T. Evaluation of D-isomers of O-11C-methyl tyrosine and O-18F-fluoromethyl tyrosine as tumor-imaging agents in tumor-bearing mice: comparison with L- and D-11C-methionine. J Nucl Med. April 2006; 47(4):679-688. In brief, the [F-18]-fluoride (34.2 GBq) was immobilized on a preconditioned QMA (Waters) cartridge (preconditioned with 5 ml 0.5M K₂CO₃ and 10 ml water). The [F-18]-fluoride was eluted with a solution of K₂CO₃ (2.7 mg) in 50 μl water and K222 (15 mg) in 950 μl acetonitrile. This solution was dried at 120° C. under vacuum and a stream of nitrogen. Additional acetonitrile (1 ml) was added and the drying step was repeated. A solution of dibromomethane (100 μl) in acetonitrile (900 μl) was added and heated at 130° C. for 5 min. The reaction was cooled and the [F-18]-fluoromethylbromide was distilled under a nitrogen flow of 50 ml/min through 4 silica cartridges into a solution of D-tyrosine (3 mg), with 10% NaOH (13.5 μl) in DMSO (1 ml). This solution was heated at 110° C. for 5 min and then cooled to 40° C. The reaction mixture was purified by HPLC (Synergi Hydro RP 4p 250×10 mm; 10% acetonitrile in water at pH 2; flow 5 ml/min). The product peak was collected, diluted with water (pH 2) and passed through a C18 Plus Environmental SPE. The SPE was washed with water pH2 (5 ml). The product was eluted with a 1:1 mixture of EtOH and water pH2 (3 ml). Starting from 34.2 GBq [F-18]-fluoride, 3.2 GBq (15% d.c.) with a specific activity of 49 GBq/μmol [F-18]-DFMT were obtained in a synthesis time of 71 minutes.

### PET/CT Imaging and Data Reconstruction

10 to 12 MBq [F-18]-fluoride or [F-18]-D-FMT were injected i.v. into the tail vein. 60 min after injection anesthesia was induced by isoflurane/O2 and twenty-minute micro-PET/computed tomography (CT) scans were obtained using an Inveon micro PET/CT scanner (Siemens).

### Histological Examination

After the PET/CT measurement, the mice were sacrificed by an overdose of isoflurane/O2. With the information from the PET images the bones which showed [F-18]-D-FMT were removed and fixed in 4% neutral-buffered formalin for several days. After fixation, decalcification in immunocal containing formic acid and routine dehydration, the samples were embedded in paraffin, and 4-6 μm thick sections were stained with hematoxylin-eosin (H&E) for microscopical examination. An immunohistochemistry for the detection of pan-cytokeratin (AE1/AE3, Abcam #ab27988, Cambridge, UK) which recognizes epitopes present in epithelial tissues was performed in order to discriminate the origin of the tumor cells: epithelial vs. nonepithelial. For differential demonstration of osteoid and collagen one slide was stained with Masson Goldner Trichrome (MGT) which stains osteoid and collagen blue green.

### Results

Detection of bone metastases by [F-18]-D-FMT was pre-clinically investigated using the 786-0/luc human renal cell adenocarcinoma bone metastasis mouse model. In in vitro experiments investigating the uptake of [F-18]-D-FMT into the 786-O/luc cells, good uptake was observed reaching 12.8% applied dose/10⁶ cells after 30 min. The luciferase gene transfected 786-O cells offered a reliable tool for following bone metastases formation in vivo by whole-body bioluminescence imaging (BLI) longitudinally. After the i.v. injection of luciferin, the luciferase containing 786-O tumor cells catalyzed the oxidation of luciferin resulting in the appearance of bioluminescence. The detection of the bioluminescence by CCD camera was used for monitoring metastasis progression and showed spread of cancer cells in the regions of hind limbs, forelimbs, spine and skull.

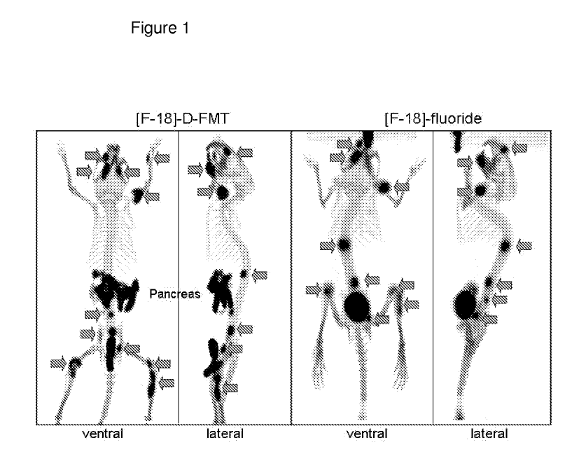

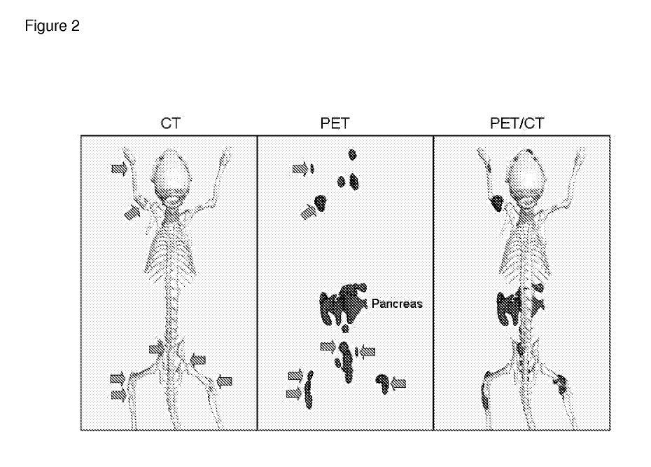

51 days after the inoculation of the 786-O/luc cells into the mice, PET/CT imaging was performed with [F-18]-fluoride (FIG. 1 right side). The images showed high accumulation in multiple osteolytic lesions in the spine, skull, forelimbs and hind limbs indicating increased mineralization compared with the uptake in healthy bone with normal appearance. The same mouse was imaged 2 weeks later (day 65) with [F-18]-D-FMT (FIG. 1 left side). The same bone lesions previously visualized with [F-18]-fluoride were visible as well as additional lesions. Thus, the localization of tumor cells monitored by [F-18]-D-FMT correlated with affected areas of the skeleton as visualized by the [F-18]-fluoride scan. There was also uptake into the pancreas of the mice. The calculation of % ID/g values based on the SUV was between 4.1 and 6.8 for the various lesions. The size of the metastases ranged from 1.5 mm to more than 7 mm in diameter. [F-18]-D-FMT showed no uptake into the healthy bone. Reconstruction of the CT and PET images by surface rendering showed that there are parts of the bones missing where the tumor cells invaded the skeleton (FIG. 2 left). The PET signal (FIG. 2 middle) showed a very specific localization which fitted into the holes in the bones, if the two images were fused (FIG. 2 right). Even very small lesions as in the shoulder blade could be visualized by PET while the CT remained inconclusive.

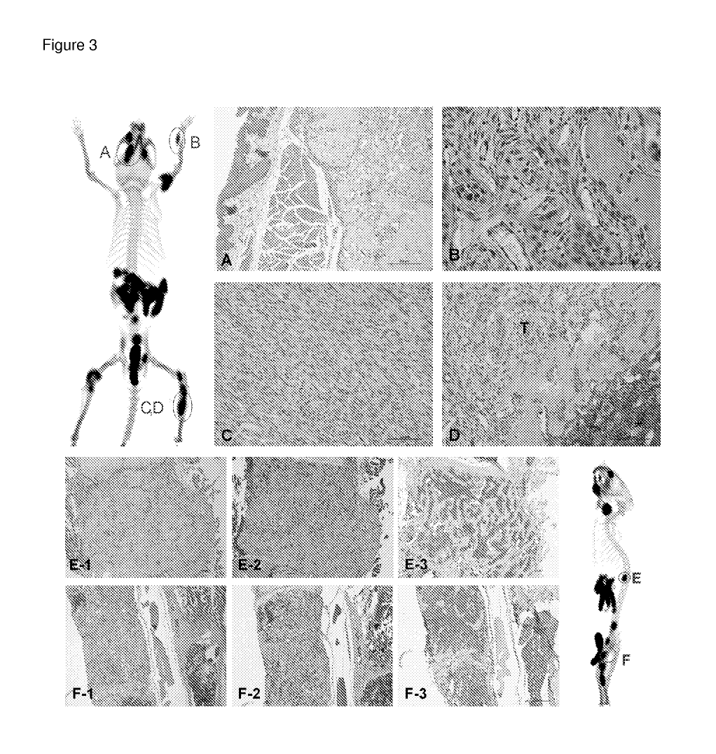

Histologically the hematopoietic cell areas are wholly replaced by tumor tissue in the medullary cavity in all samples collected after the PET/CT imaging. The proliferating cells were large pleomorphic, with abounded cytoplasm and round dense nuclei, in other areas spindle-shaped cells separated by a moderate amount of collganous matrix were more predominant (FIG. 3). A moderate number of mitotic figures were present (0-3 at 40×). Additionally, in some samples multinucleated giant cells were present. An essential feature of the tumors was that lysis of normal bone occurred simultaneously with the formation of new osteoid, which stained blue green with MTG (FIG. 3). The tumor cells were positive for pan-cytokeratin, which confirmed that they are of epithelial origin.

In the experiments, it is clearly shown that [F-18]-D-FMT is able to detect bone metastases in a nude mouse model.

The areas of the bone, which showed accumulation of [F-18]-D-FMT were removed and histologically examined. Tumor cells were detected which had invaded the bones and which are most likely responsible for the [F-18]-D-FMT accumulation. In addition, Tsukada et al (Tsukada H, Sato K, Fukumoto D, Nishiyama S, Harada N, Kakiuchi T. Evaluation of D-isomers of O-11C-methyl tyrosine and O-18F-fluoromethyl tyrosine as tumor-imaging agents in tumor-bearing mice: comparison with L- and D-11C-methionine. J Nucl Med. April 2006; 47(4):679-688.) it was demonstrated in a Turpentine-induced inflammation model, that [F-18]-D-FMT shows no uptake in inflammatory muscle tissue whereas FDG was taken up in inflammatory muscle tissue.

[F-18]-fluoride reflects an unspecific uptake into regenerating and remineralizing bone, also the larger bones (spine, legs) as well as the joints showed [F-18]-fluoride uptake. In contrast to [F-18]-fluoride, osteoblastic activity is not detected by [F-18]-D-FMT.

Comparison of the PET/CT scans of [F-18]-fluoride and the [F-18]-D-FMT scan showed that [F-18]-D-FMT accumulated in all bone metastases imaged by [F-18]-fluoride but not in bone wherein osteoblastic activity was showed with [F-18]-fluoride.

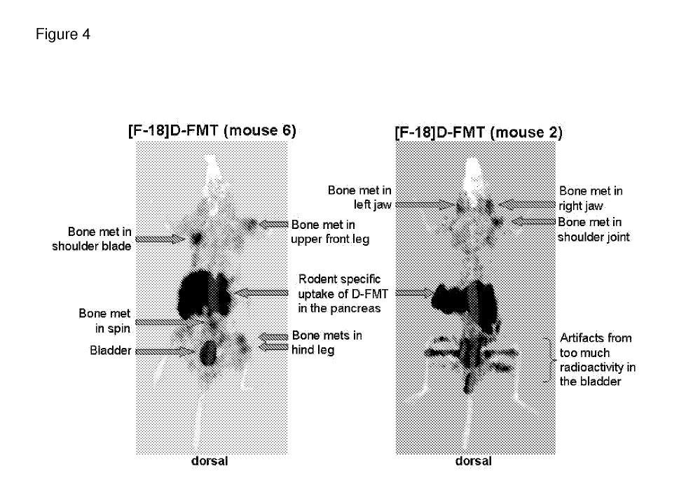

25 days after the inoculation of the MDA-MB231SA/luc cells into the mice, PET/CT imaging was performed with [F-18]-D-FMT as a second bone metastases model using a breast carcinoma cell line MDA-MB231SA/luc which is an established model for the formation of bone metastases (Mbalaviele G, Dunstan C R, Sasaki A, Williams P J, Mundy G R, Yoneda T. E-cadherin expression in human breast cancer cells suppresses the development of osteolytic bone metastases in an experimental metastasis model. Cancer Res 1996; 56:4063-70.). [F-18]-D-FMT also showed uptake into the bone metastases (FIG. 4). To conclude, [F-18]-D-FMT is useful for the detection of bone metastases.

1. A compound of general formula (I) for imaging bone metastases wherein compound of general formula (I) is 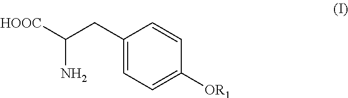
R₁ is —CH₂—F¹⁸, —CH₂—CH₂—F¹⁸ or —CH₂—CH₂—CH₂—F¹⁸; and
single isomers, enantiomers, stereoisomers, stereoisomeric mixtures or mixtures thereof and pharmaceutically acceptable salts thereof.

2. A compound according to claim 1 wherein compound of general formula (I) is

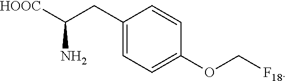

3. A compound according to claim 1 wherein the bone proliferative disease is characterised by the presence of bone metastases.

4. A method for differentiating bone metastatic disease from bone non-metastatic disease in a mammal wherein bone non-metastatic disease is a benign bone pathology comprised from the group of back pains, focal changes in bones, trauma, reconstructive surgery, bone grafts, metabolic bone disease or osteoporosis, which comprises using a compound of formula (I) as an imaging agent; 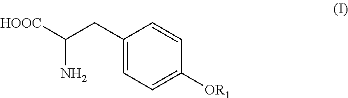
R₁ is —CH₂—F¹⁸, —CH₂—CH₂—F¹⁸ or —CH₂—CH₂—CH₂—F¹⁸ and
single isomers, enantiomers, stereoisomers, stereoisomeric mixtures or mixtures thereof and pharmaceutically acceptable salts thereof.

5. A composition comprising a compound of the general formula (I) according to claim 1 and pharmaceutically acceptable carrier or diluent wherein the compounds of the general formula (I) is an imaging tracer for imaging bone metastases.

6. A kit comprising a sealed vial containing a predetermined quantity of a compound having general chemical Formula (I) according to claim 1 and suitable salts of inorganic or organic acids thereof, hydrates, complexes, esters, amides, and solvates thereof wherein the compounds of the general formula (I) is an imaging tracer for imaging bone metastases.
